(19) United States
(12) Patent Application Publication
(10) Pub. No.: US 2005/0070919 A1
(43) Pub. Date: Mar. 31, 2005

(54) Device for guiding insertion of a medical device into the spinal epidural space and method therefor

(76) Inventor: Daniel M. Lieberman, Phoenix, AZ (US)

Correspondence Address: WEISS & MOY PC, 4204 NORTH BROWN AVENUE, SCOTTSDALE, AZ, 85251, US

(21) Appl. No.: 10/947,798

(22) Filed: Sep. 23, 2004

Related U.S. Application Data
(60) Provisional application No. 60/506,446, filed on Sep. 26, 2003.

(51) Int. Cl. A61F 11/00

(52) U.S. Cl. 606/108

(57) ABSTRACT

The device of the present invention guides the insertion of a medical device into the spinal epidural space of a vertebral column. Preferably, the device will be dimensioned to guide insertion of a medical device through a tubular retractor. Further preferably, the device may be used by surgeons to dissect the dura mater away from the lamina. Still further preferably, the device would protect the spinal cord from injury during insertion of a spinal cord stimulator by guiding the spinal cord stimulator through the epidural space at an angle substantially parallel to the spinal cord.

### CROSS-REFERENCE TO RELATED APPLICATIONS

This application claims priority to a corresponding provisional application U.S. Ser. No. 60/506,446, filed Sep. 26, 2003 in the name of the applicant of this application.

### BACKGROUND OF THE INVENTION

Spinal cord stimulators have been used to treat patients who experience chronic and severe back pain. A spinal cord stimulator is a device that stimulates the spinal cord by tiny electrical impulses from small electrical wires placed on the spinal cord itself. These electrical impulses interrupt nerve conduction (i.e. the conduction of pain signals) to the brain.

As shown in FIGS. 4-6, the vertebral column 100 (see FIGS. 4 and 6) is made up of a series of vertebrae 110, each vertebra 110 consisting of a body 116 (see FIG. 5) and a vertebral arch 118 (see FIG. 5) consisting of the laminar bones. Together, the body 116, pedicles, and lamina 120 define an opening called the vertebral canal 112 (see FIG. 6), through which the spinal cord 122 (see FIGS. 5 and 6) passes.

Within the vertebral canal 112, the spinal cord 122 is surrounded and protected by the dura mater 124 (see FIGS. 5 and 6) and lamina 120 (see FIGS. 5 and 6). The dura mater 124 is a thin, leathery membrane which surrounds the spinal cord 122, and the lamina 120 is a flat bony plate that helps form the vertebral arch 118 of each vertebra 110. Between the dura mater 124 and the bony lamina 120 is the epidural space 126 (see FIGS. 5 and 6) filled with a soft padding of fat and a network of veins.

The method of insertion of a spinal cord stimulator depends upon its width. The width of existing spinal cord stimulators varies between 1 mm and 8 mm and the height varies between 1 mm and 3 mm. Smaller, narrower leads and cannulas are sometimes inserted into the epidural space by placement of a Tuohy needle, which has a curved tip, into the epidural space. The curve of the Tuohy needle tip guides the lead into the epidural space, parallel to the spinal cord, so as to avoid injury to the spinal cord 122 by the lead during insertion.

For wider spinal cord stimulators 200 (see FIGS. 1A, 1, 4, and 6), however, surgeons must perform a laminotomy or a limited laminectomy, wherein the lamina 120 is partially removed in order to create space for insertion of the spinal cord stimulator 200 into the epidural space 126. After the laminotomy or laminectomy, a paddle probe (a curved, flat probe with length and width similar to the spinal cord stimulator 200) is used to dissect the dura mater 124 away from the remaining lamina 120 distal to the site of the laminotomy or laminectomy. Subsequently, the surgeon may manually direct the spinal cord stimulator 200 along the tract dissected by the paddle probe. Although the paddle probe is used to dissect the dura mater 124 away from the lamina 120, it does not facilitate or guide the spinal cord stimulator 200 during insertion.

Tubular retractors 300 (see FIGS. 4-6) are used to permit minimally invasive access to the vertebral canal 112 for spinal surgery. The tubular retractor 300 is positioned in the vertebral column 100 by sequential dilation of the paraspinous muscles. Once in position, the tubular retractor 300 is held in position by an arm attached to the operating bed.

Although tubular retractors 300 offer a minimally invasive exposure of the epidural space 126, the length of the tubular retractor 300 (typically between 3 cm and 9 cm) makes it difficult for the surgeon to manually guide wider spinal cord stimulators 200 into the epidural space 126. Additionally, the paddle probe has too large of an angle to fit conveniently through a tubular retractor 300.

Therefore, a need existed for a device and method for guiding insertion of a medical device into the spinal epidural space of a vertebral column. Preferably, the device will be dimensioned to guide insertion of a medical device through a tubular retractor. Further preferably, the device may be used by surgeons to dissect the dura mater away from the lamina. Still further preferably, the device would protect the spinal cord from injury during insertion of a spinal cord stimulator by directing the spinal cord stimulator through the epidural space at an angle substantially parallel to the spinal cord.

### SUMMARY OF THE INVENTION

An object of the present invention is to provide a device for guiding insertion of a medical device into the spinal epidural space of the vertebral column.

It is a further object of the present invention to provide a device for guiding insertion of a medical device through a tubular retractor.

It is a further object of the present invention to provide a device that may be used to dissect the dura mater away from the lamina of the vertebra.

It a still further object of the present invention to provide a device that will protect the spinal cord from injury during insertion of a spinal cord stimulator by guiding the spinal cord stimulator through the epidural space at an angle substantially parallel to the spinal cord.

### BRIEF DESCRIPTION OF THE PREFERRED EMBODIMENTS

In accordance with one embodiment of the present invention, a device for guiding insertion of a medical device into a target area of a vertebrate is disclosed, comprising a substantially J-shaped angled probe comprising a first end, a shaft portion coupled to and extending from the first end, a substantially curved neck portion coupled to and extending at an angle from the shaft portion, the shaft portion dimensioned to guide a medical device from the first end of the angled probe through the substantially curved neck portion, a second end coupled to the substantially curved neck portion and disposed opposite the first end, the second end dimensioned to guide the medical device to the target area of the vertebrate at an angle substantially perpendicular to the shaft portion of the angled probe, and at least one side rail coupled to and extending at an angle substantially perpendicular from at least one of the neck portion and the second end.

In accordance with another embodiment of the present invention, a device for guiding insertion of a medical device into a target area of a vertebrate is disclosed, comprising a substantially J-shaped angled probe comprising a first end, a shaft portion coupled to and extending from the first end, a substantially curved neck portion coupled to and extending at an angle from the shaft portion, the shaft portion dimensioned to guide a medical device from the first end of the angled probe through the substantially curved neck portion, a second end coupled to the substantially curved neck portion and disposed opposite the first end, the second end dimensioned to guide the medical device to the target area of the vertebrate at an angle substantially perpendicular to the shaft portion of the angled probe, and a roof portion coupled to and enclosing at least a portion of at least one of the substantially curved neck portion and the second end.

In accordance with yet another embodiment of the present invention, a method for guiding insertion of a medical device into a target area of a vertebrate is disclosed, comprising, in combination, the steps of providing a substantially J-shaped angled probe having a first end, a shaft portion coupled to and extending from the first end, a substantially curved neck portion coupled to and extending at an angle from the shaft portion, a second end coupled to the substantially curved neck portion and disposed opposite the first end, and at least one side rail coupled to and extending at an angle substantially perpendicular from at least one of the neck portion and the second end, inserting the angled probe into the target area of the vertebrate, guiding a medical device substantially downwardly along the angled probe, and inserting the medical device into the target area of the vertebrate at an angle substantially parallel to the target area.

In accordance with still another embodiment of the present invention, a method for guiding insertion of a medical device into a target area of a vertebrate is disclosed, comprising, in combination, the steps of providing a substantially J-shaped angled probe having a first end, a shaft portion coupled to and extending from the first end, a substantially curved neck portion coupled to and extending at an angle from the shaft portion, a second end coupled to the substantially curved neck portion and disposed opposite the first end, and a roof portion coupled to and enclosing at least a portion of at least one of the substantially curved neck portion and the second end, inserting the angled probe into the target area of the vertebrate, guiding the medical device substantially downwardly along the angled probe between the roof portion and at least a portion of at least one of the substantially curved neck portion and the second end, and inserting the medical device into the target area of the vertebrate at an angle substantially parallel to the target area.

In accordance with still another embodiment of the present invention, a method for guiding insertion of a spinal cord stimulator into a spinal epidural space through a tubular retractor is disclosed, comprising, in combination, the steps of providing a tubular retractor, positioning the tubular retractor to allow access to the spinal epidural space, providing a substantially J-shaped angled probe having a first end, a shaft portion coupled to and extending from the first end, a substantially curved neck portion coupled to and extending at an angle from the shaft portion, a second end coupled to the substantially curved neck portion and disposed opposite the first end, and at least one side rail coupled to and extending at an angle substantially perpendicular from at least one of the neck portion and the second end, inserting the angled probe through an aperture defined by the tubular retractor, penetrating the spinal epidural space with the second end of the angled probe, dissecting at least a portion of the dura mater from the lamina with the second end of the angled probe, providing a spinal cord stimulator, and dissecting the dura mater from the remaining lamina while the spinal cord stimulator being guided substantially downwardly along the angled probe into the spinal epidural space at an angle substantially parallel to a spinal cord.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

FIG. 1A is a top view of a spinal cord stimulator.

FIG. 1 is a perspective view of the spinal cord stimulator of FIG. 1A being guided along one embodiment of an angled probe in accordance with the present invention.

FIG. 2 is a perspective view of another embodiment of an angled probe in accordance with the present invention, showing a first and second side groove coupled to and extending from the second end through the curved neck portion of the probe.

FIG. 3 is a perspective view of yet another embodiment of an angled probe in accordance with the present invention, showing a shorter first and second side groove coupled to and extending from the end of the curved neck portion to the second end of the probe.

FIG. 4 is a perspective view of yet another embodiment of an angled probe in accordance with the present invention being inserted through a tubular retractor positioned within a vertebral column.

FIG. 5 is a top view of a typical lumbar vertebra showing a cross-section of a spinal cord and surrounding dura mater, epidural space, and lamina. Yet another embodiment of an angled probe is also shown being inserted into a tubular retractor positioned within the vertebral column.

FIG. 6 is a side, cross-sectional view of a typical vertebral column showing the spinal cord stimulator of FIG. 1A being guided along an angled probe through the tubular retractor and into the epidural space at an angle substantially perpendicular to the spinal cord.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

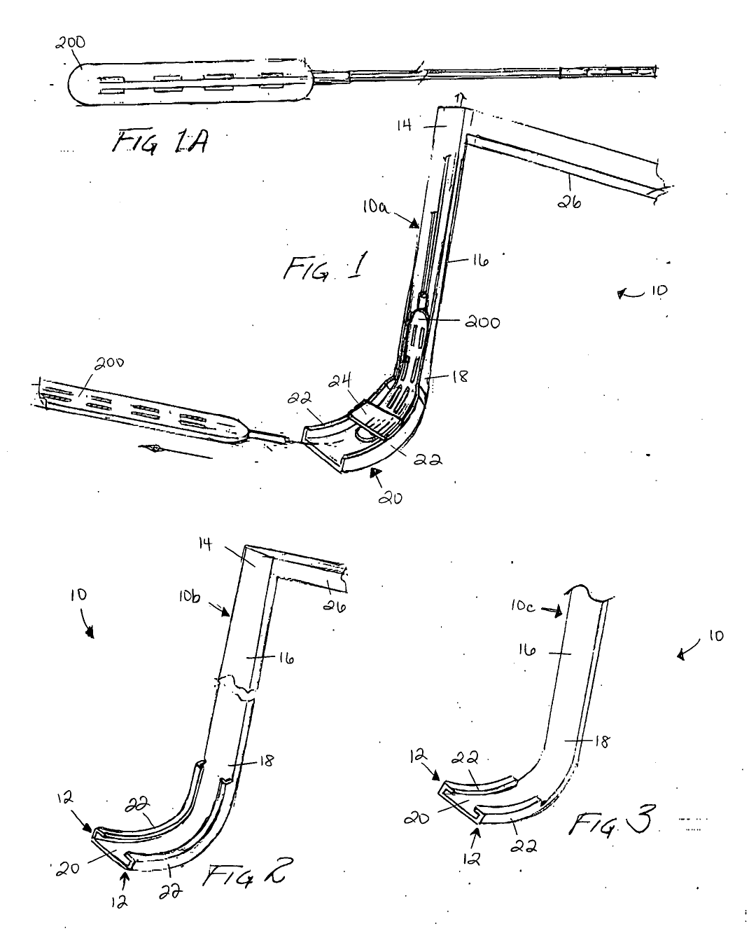

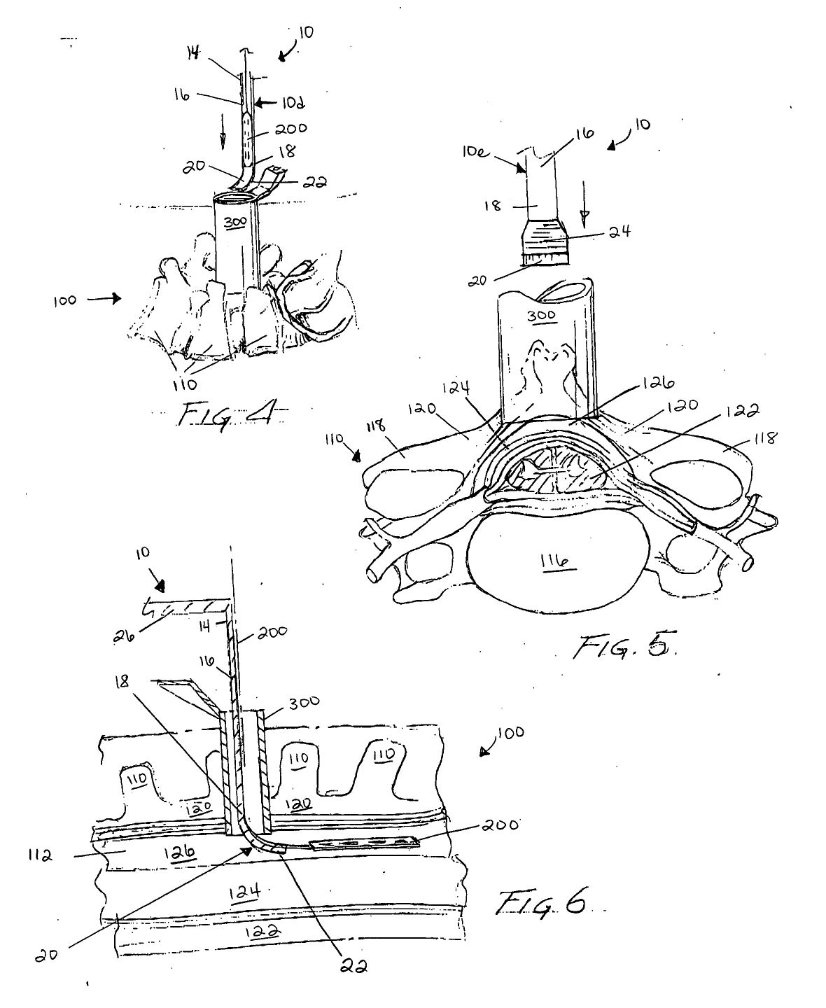

Referring to FIGS. 1-6, various embodiments of an angled probe, referred to generically as angled probe 10, are disclosed. The angled probe 10 is for guiding insertion of a medical device into a target area of a vertebrate. The angled probe 10 is substantially J-shaped and is preferably dimensioned to guide insertion of a spinal cord stimulator 200 (see FIGS. 1A, 1, 4 and 6) into a spinal epidural space 126 (see FIGS. 5 and 6) of a vertebral column 100 (see FIGS. 4 and 6) through a tubular retractor 300 (see FIGS. 4-6), although it should be clearly understood that substantial benefit may be derived from an alternative configuration of the angled probe 10 in which the angled probe 10 is dimensioned to guide insertion of an alternate medical device (e.g. a lead, a probe, or a catheter) into an alternate area of the vertebral column 100. It should be further understood that substantial benefit may be derived from an alternative configuration of the angled probe 10 in which the angled probe 10 is dimensioned to be used without a tubular retractor 300. Furthermore, although the angled probe 10 is preferably dimensioned to be used in surgery on human beings, it should be clearly understood that substantial benefit could be derived from using the angled probe 10 on other mammals or vertebrates.

Preferably, the angled probe 10 is dimensioned to be used with a spinal cord stimulator 200 having a length of between 4 cm and 6 cm, a width of between 1 mm and 8 mm, and a height of between 1 mm and 3 mm. It should be clearly understood, however, that substantial benefit may be derived from an angled probe 10 that is dimensioned to be used with a spinal cord stimulator 200 having a length, width, or height that deviates, even substantially, from the length, width, and height of the preferred spinal cord stimulator 200. And although it is preferred that the angled probe 10 be dimensioned to guide insertion of the spinal cord stimulator 200 through a tubular retractor 300 having a length of between 3 cm and 10 cm it should be clearly understood that substantial benefit may be derived from the angled probe 10 being dimensioned to be used with a tubular retractor 300 having a length that deviates, even substantially, from the length of the preferred tubular retractor 300.

Still referring to FIGS. 1-6, the angled probe 10 comprises a first end 14 (see FIGS. 1, 2, 4, and 6), a shaft portion 16 coupled to and extending from the first end 14, a substantially curved neck portion 18 coupled to and extending at an angle from the shaft portion 16, and a second end 20 coupled to the curved neck portion 18 and dimensioned to guide the spinal cord stimulator 200 into the spinal epidural space 126 at an angle substantially perpendicular to the spinal cord 122. Preferably the angled probe 10 further comprises a handle 26 (see FIGS. 1, 2, and 6) coupled substantially perpendicular to the first end 14 of the angled probe 10 so that the handle 26 may be held at an angle substantially perpendicular to the tubular retractor 300, thereby maintaining an open view down the tubular retractor 300 and into the spinal epidural space 126, although it should be clearly understood that substantial benefit could be derived from an alternative configuration of the present invention in which no handle 26 is used.

Referring specifically to FIG. 1, a first embodiment of the angled probe 10, hereinafter referred to as angled probe 10a, is shown. In this embodiment, the angled probe 10a has two side rails 22 coupled to and extending substantially perpendicular from the neck portion 18 and the second end 20. Furthermore, a roof portion 24 is coupled to the two side rails 22 and encloses at least a portion of the substantially curved neck portion 18. Preferably, the spinal cord stimulator 200 will be guided substantially downwardly along the angled probe 10 between the substantially curved neck portion 18 and the roof portion 24.

Referring now to FIGS. 2 and 3, FIG. 2 discloses a second embodiment of the angled probe 10, hereinafter referred to as angled probe 10b and FIG. 3 discloses a third embodiment of the angled probe 10, hereinafter referred to as angled probe 10c. Angled probe 10b has two side rails 22 coupled to and extending substantially perpendicular from the neck portion 18 and the second end 20, whereas angled probe 10c has two side rails 22 coupled to and extending substantially perpendicular from only the second end 20. Both angled probe 10b and angled probe 10c, however, have two side rails 22 that curve substantially inwardly so as to form two side grooves 12. Preferably, the spinal cord stimulator 200 will be guided substantially downwardly along the angled probe 10 and between the two side grooves 12.

Referring now to FIG. 4, a fourth embodiment of the angled probe 10, hereinafter referred to as angled probe 10d, is disclosed. In this embodiment, the angled probe 10d has two side rails 22 coupled to and extending substantially perpendicular from the neck portion 18 and the second end 20. Preferably, each side rail 22 has a tapered height, although it should be understood that substantial benefit may be derived from side rails 22 that are not tapered. It should be further understood that substantial benefit may be derived from an angled probe 10 having only one side rail 22. And although it is preferred that each side rail 22 rise to a height between approximately 0.05 mm and 3 mm at the second end 20 of the angled probe 10d, it should be clearly understood that each side rail 22 may rise to an alternate height, so long as each side rail 22 is dimensioned to guide the spinal cord stimulator 200 steadily along the angled probe 10.

Referring now to FIG. 5, a fifth embodiment of the angled probe 10, hereinafter referred to as angled probe 10e, is disclosed. In this embodiment, the angled probe 10e has a roof portion 24 coupled to and enclosing both the substantially curved neck portion 18 and the second end 20. However, it should be clearly understood that substantial benefit may be derived from the roof portion 24 covering and enclosing only the substantially curved neck portion 18. Furthermore, it should be clearly understood that substantial benefit may be derived from the roof portion 24 covering and enclosing only the second end 20.

While, in the various embodiments described above, the angled probe 10 is shown having various combinations of side rails 22 of varying dimensions, side grooves 12 of varying dimensions, and roof portions 24 of varying dimensions, it should be clearly understood that substantial benefit could be derived from alternative configurations of the angled probe 10 in which other combinations not described here are used. For example, substantial benefit may be derived from an angled probe 10 having both side grooves 12 as well as a roof portion 24.

### Statement of Operation

In order to implant a spinal cord stimulator 200, a laminotomy or a limited laminectomy is performed. Then the angled probe 10 is passed through the tubular retractor 300 and inserted into the spinal epidural space 126 of the vertebral canal 112. The angled probe 10 is used to dissect a portion of the dura mater 124 away from the lamina 120. Then, the spinal cord stimulator 200 is used to dissect the dura mater 124 from the remaining lamina 120 distal to the site of the laminotomy or laminectomy, while the spinal cord stimulator 200 is guided substantially downwardly along the angled probe 10 and into the spinal epidural space 126 at an angle substantially parallel to the spinal cord 122.

While the invention has been particularly shown and described with reference to preferred embodiments thereof, it will be understood by those skilled in the art that the foregoing and other changes in form and details may be made therein without departing from the spirit and scope of the invention. For example, although the angled probe is intended to guide insertion of medical devices into the epidural space of a vertebral column, it should be clearly understood that substantial benefit may be derived from its use in guiding insertion of medical devices into alternate target areas of the body.

1. A device for guiding insertion of a medical device into a target area of a vertebrate comprising an angled probe, said angled probe being substantially J-shaped and comprising:
a first end;
a shaft portion coupled to and extending from said first end;
a substantially curved neck portion coupled to and extending at an angle from said shaft portion, said shaft portion dimensioned to guide a medical device from said first end of said angled probe through said substantially curved neck portion;
a second end coupled to said substantially curved neck portion and disposed opposite said first end, said second end dimensioned to guide said medical device to said target area of said vertebrate at an angle substantially perpendicular to said shaft portion of said angled probe; and
at least one side rail coupled to and extending at an angle substantially perpendicular from at least one of said neck portion and said second end.

2. The device of claim 1 further comprising a handle coupled to said first end of said angled probe and dimensioned to be gripped by a person.

3. The device of claim 2 wherein said handle being coupled substantially perpendicular to said first end of said angled probe.

4. The device of claim 1 wherein said vertebrate being a mammal.

5. The device of claim 1 wherein said vertebrate being a human being.

6. The device of claim 1 wherein said target area being spinal epidural space of a vertebral column.

7. The device of claim 1 wherein said medical device being at least one of a lead, a probe, a catheter, and a spinal cord stimulator.

8. The device of claim 7 wherein said spinal cord stimulator having:
a length of between 4 cm and 6 cm;
a width of between 1 mm and 8 mm; and
a height of between 1 mm and 3 mm.

9. The device of claim 1 wherein said angled probe being adapted to be inserted through an aperture defined by a tubular retractor.

10. The device of claim 9 wherein said tubular retractor having a length of between 3 cm and 10 cm.

11. The device of claim 3 wherein said angled probe being adapted to be inserted through an aperture defined by a tubular retractor, said handle of said angled probe being dimensioned to be held at an angle substantially perpendicular to said tubular retractor so as to maintain an open view down said aperture defined by said tubular retractor.

12. The device of claim 1 further comprising a roof portion coupled to and enclosing at least a portion of said substantially curved neck portion of said angled probe.

13. The device of claim 1 wherein said at least one side rail having a tapered height.

14. The device of claim 13 wherein said at least one side rail rising to a height between approximately 0.05 mm and 3 mm at said second end of said angled probe.

15. The device of claim 1 wherein said at least one side rail curving substantially inwardly so as to form at least one side groove.

16. The device of claim 1 wherein said at least one side rail being two side rails coupled to and extending at an angle substantially perpendicular from said neck portion and said second end.

17. The device of claim 16 further comprising a roof portion coupled to said two side rails and enclosing at least a portion of at least one of said substantially curved neck portion and said second end.

18. The device of claim 1 wherein said first end and said shaft portion and said substantially curved neck portion and said second end and said at least one side rail comprise a one-piece assembly.

19. A device for guiding insertion of a medical device into a target area of a vertebrate comprising an angled probe, said angled probe being substantially J-shaped and comprising:
a first end;
a shaft portion coupled to and extending from said first end;
a substantially curved neck portion coupled to and extending at an angle from said shaft portion, said shaft portion dimensioned to guide a medical device from said first end of said angled probe through said substantially curved neck portion;
a second end coupled to said substantially curved neck portion and disposed opposite said first end, said second end dimensioned to guide said medical device to said target area of said vertebrate at an angle substantially perpendicular to said shaft portion of said angled probe; and
a roof portion coupled to and enclosing at least a portion of at least one of said substantially curved neck portion and said second end.

20. The device of claim 19 further comprising a handle coupled to said first end of said angled probe and dimensioned to be gripped by a person.

21. The device of claim 20 wherein said handle being coupled substantially perpendicular to said first end of said angled probe.

22. The device of claim 19 wherein said vertebrate being a mammal.

23. The device of claim 19 wherein said vertebrate being a human being.

24. The device of claim 19 wherein said target area being a spinal epidural space of a vertebral column.

25. The device of claim 19 wherein said medical device being at least one of a lead, a probe, a catheter, and a spinal cord stimulator.

26. The device of claim 25 wherein said spinal cord stimulator having:
a length of between 4 cm and 6 cm;
a width of between 1 mm and 8 mm; and
a height of between 1 mm and 3 mm.

27. The device of claim 19 wherein said angled probe being adapted to be inserted through an aperture defined by a tubular retractor.

28. The device of claim 27 wherein said tubular retractor having a length of between 3 cm and 10 cm.

29. The device of claim 21 wherein said angled probe being adapted to be inserted through an aperture defined by a tubular retractor, said handle of said angled probe being dimensioned to be held at an angle substantially perpendicular to said tubular retractor so as to maintain an open view down said aperture defined by said tubular retractor.

30. The device of claim 19 further comprising at least one side rail coupled to and extending at an angle substantially perpendicular from at least one of said neck portion and said second end.

31. The device of claim 30 wherein said at least one side rail having a tapered height.

32. The device of claim 31 wherein said at least one side rail rising to a height between approximately 0.05 mm and 3 mm at said second end of said angled probe.

33. The device of claim 30 wherein said at least one side rail curving substantially inwardly so as to form at least one side groove.

34. The device of claim 30 wherein said at least one side rail being two side rails coupled to and extending at an angle substantially perpendicular from said neck portion and said second end.

35. The device of claim 34 wherein said roof portion being coupled to said two side rails and enclosing at least a portion of at least one of said substantially curved neck portion and said second end.

36. The device of claim 19 wherein said first end and said shaft portion and said substantially curved neck portion and said second end and said roof portion comprise a one-piece assembly.

37. A method for guiding insertion of a medical device into a target area of a vertebrate comprising, in combination, the steps of:
providing a substantially J-shaped angled probe having:
a first end;
a shaft portion coupled to and extending from said first end;
a substantially curved neck portion coupled to and extending at an angle from said shaft portion;
a second end coupled to said substantially curved neck portion and disposed opposite said first end; and
at least one side rail coupled to and extending at an angle substantially perpendicular from at least one of said neck portion and said second end;
inserting said angled probe into said target area of said vertebrate;
guiding a medical device substantially downwardly along said angled probe; and
inserting said medical device into said target area of said vertebrate at an angle substantially parallel to said target area.

38. The method of claim 37 further comprising the steps of:
providing a handle coupled to said first end of said angled probe; and
gripping said handle while inserting said angled probe into said target area of said vertebrate.

39. The method of claim 37 further comprising the step of inserting said angled probe into an epidural space of a vertebral column.

40. The method of claim 37 further comprising the steps of:
providing a tubular retractor;
positioning said tubular retractor so as to allow access to said target area; and
inserting said angled probe through an aperture defined by said tubular retractor so that said second end of said angled probe being proximate said target area.

41. The method of claim 37 wherein said medical device being at least one of a lead, a probe, a catheter, and a spinal cord stimulator.

42. The method of claim 37 further comprising the steps of:
providing a roof portion coupled to and enclosing at least a portion of at least one of said substantially curved neck portion and said second end of said angled probe; and
guiding said medical device substantially downwardly along said angled probe between said substantially curved neck portion and said roof portion; and
inserting said medical device into said target area.

43. A method for guiding insertion of a medical device into a target area of a vertebrate comprising, in combination, the steps of:
providing a substantially J-shaped angled probe having:
a first end;
a shaft portion coupled to and extending from said first end;
a substantially curved neck portion coupled to and extending at an angle from said shaft portion;
a second end coupled to said substantially curved neck portion and disposed opposite said first end; and
a roof portion coupled to and enclosing at least a portion of at least one of said substantially curved neck portion and said second end;
inserting said angled probe into said target area of said vertebrate;
guiding said medical device substantially downwardly along said angled probe between said roof portion and at least a portion of at least one of said substantially curved neck portion and said second end; and
inserting said medical device into said target area of said vertebrate at an angle substantially parallel to said target area.

44. The method of claim 43 further comprising the steps of:
providing a handle coupled to said first end of said angled probe; and
gripping said handle while inserting said angled probe into said target area of said vertebrate.

45. The method of claim 43 further comprising the step of inserting said angled probe into an epidural space of a vertebral column.

46. The method of claim 43 further comprising the steps of:
providing a tubular retractor;
positioning said tubular retractor so as to allow access to said target area; and
inserting said angled probe through an aperture defined by said tubular retractor so that said second end of said angled probe being proximate said target area.

47. The method of claim 43 wherein said medical device being at least one of a lead, a probe, a catheter, and a spinal cord stimulator.

48. The method of claim 43 further comprising the steps of:
at least one side rail coupled to and extending at an angle substantially perpendicular from at least one of said neck portion and said second end; and
guiding said medical device substantially downwardly along said at least one side rail; and
inserting said medical device into said target area.

49. A method for guiding insertion of a spinal cord stimulator into a spinal epidural space through a tubular retractor comprising, in combination, the steps of:
providing a tubular retractor;
positioning said tubular retractor to allow access to a spinal epidural space;
providing a substantially J-shaped angled probe having:
a first end;
a shaft portion coupled to and extending from said first end;
a substantially curved neck portion coupled to and extending at an angle from said shaft portion;
a second end coupled to said substantially curved neck portion and disposed opposite said first end; and
at least one side rail coupled to and extending at an angle substantially perpendicular from at least one of said neck portion and said second end;
inserting said angled probe through an aperture defined by said tubular retractor;
penetrating said spinal epidural space with said second end of said angled probe;
dissecting at least a portion of the dura mater from the lamina with said second end of said angled probe;
providing a spinal cord stimulator; and
dissecting the dura mater from the remaining lamina while said spinal cord stimulator being guided substantially downwardly along said angled probe into said spinal epidural space at an angle substantially parallel to a spinal cord.
